(19) United States
(12) Patent Application Publication
(10) Pub. No.: US 2003/0212304 A1
(43) Pub. Date: Nov. 13, 2003

(54) Systems and method for practicing counter-cardiac retrograde perfusion

(76) Inventor: Omar M. Lattouf, Atlanta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/389,415

(22) Filed: Mar. 14, 2003

Related U.S. Application Data
(60) Provisional application No. 60/364,564, filed on Mar. 16, 2002.

(51) Int. Cl. A61N 1/362

(52) U.S. Cl. 600/17

(57) ABSTRACT

Disclosed are systems and methods for performing counter-cardiac retrograde perfusion. Perfusion is performed in a counter-cardiac cycle such that oxygenated blood is urged through a patient vein associated with an organ to be provided with the blood during diastole only.

### CROSS REFERENCE TO RELATED APPLICATION

This application is claims benefit to U.S. Provisional Application filed Mar. 16, 2002, having serial No. 60/364,564.

### BACKGROUND

Under various conditions, oxygenated blood, or a necessary quantity of oxygenated blood, cannot reach a given organ. For example, during or after a stroke, blockage of a carotid artery may result in insufficient oxygen supply to the brain, thereby causing damage to the brain, such as an infarct, or even cerebral death. To cite another example, if a renal artery is blocked or constricted for some reason, the oxygen needed by the kidney supplied by the renal artery may not reach the kidney. Such a condition may occur, for instance, when cardiac surgery is being performed and the patient's blood pressure is reduced and/or artery-constrictive pharmacologic agents, such as vassopressive medications, are administered. In such a case, the kidney, particularly if already in a weakened condition prior to the surgery, can fail.

Although various therapies exist for dealing with such reduced supply of oxygenated blood such as medical supportive care, heparinization, thrombolytic therapy, or stenting of occlusive lesions, the availability of such therapies are offered by a relatively limited number of medical facilities. In situations in which treatment is needed immediately to preserve organ life (e.g., in the case of an acute or impending stoke), patients remote from such facilities are at high-risk of death or disabling morbidity.

In view of the seriousness of the consequences when an organ, and in particular the brain, is starved of oxygen, physicians have attempted to develop techniques with which oxygenated blood can be provided to the organ. One such technique is known as retrograde perfusion and, when pertaining to the brain, is referred to retrograde cerebral perfusion (RCP). RCP involves siphoning oxygenated blood from the patient's arterial system and supplying that blood to the venous system associated with the organ at tissue in a reversed or “retrograde” direction. Accordingly, oxygenated blood is passed through a vein, for example through an internal jugular vein, in a direction opposite to the normal flow of blood through the vein. By doing so, oxygenated blood is forced into the organ at issue (e.g., brain) and, theoretically, life-sustaining oxygen is provided to the organ. RCP has been shown to be a valuable method for providing a longer period of brain protection during deep hypothermic circulatory arrest (DHTCA).

Although retrograde perfusion is useful in treating patients in situations in which arterial blood flow to the organ is reduced, present techniques are limited and/or disadvantageous in some respects. One reason for this is the fact that the oxygenated blood from the patient's arterial system is provided, i.e. pumped, into the venous system continuously at a constant rate without accounting for diastole and systole. Accordingly, blood is pumped in a retrograde direction into the vein both during heart contraction (systole), when blood is pumped through the vein, and during heart relaxation (diastole). This condition results in increased blood pressure in the capillaries that serve as a junction between the arterial system and the venous system for the organ. Such increased capillary pressure can result in brain edema and hemorrhage.

Continuous flow of blood provided into the patient's vein can produce other problems. One such problem is edema. Specifically, when oxygenated blood is provided to the organ continuously, the draining function provided by the venous system is interrupted, therefore resulting in blood pooling within the organ that can cause swelling that can irreparably damage the organ.

Because of the drawbacks associated with known techniques, it can be appreciated that improved methods for performing retrograde perfusion, as well as systems for performing those methods, would be desirable.

### SUMMARY OF THE DISCLOSURE

Disclosed are systems and methods for performing counter-cardiac retrograde perfusion. In one embodiment, a system for performing counter-cardiac retrograde perfusion includes a venous catheter configured for insertion into a vein that serves an organ to be supplied with oxygenated blood, an arterial catheter configured for insertion into an artery that supplies oxygenated blood, a monitoring device configured to monitor cardiac function of the patient, and a pumping device in electrical communication with the monitoring device, the pumping device being configured to siphon blood from the artery via the arterial catheter and pump it into the vein via the venous catheter, wherein blood is pumped into the vein in accordance with cardiac function information provided by the monitoring device such that blood is pumped into the vein in a counter-cardiac manner only during diastole.

In another embodiment, a system for performing counter-cardiac retrograde perfusion includes a venous catheter configured for insertion into a vein that serves an organ to be supplied with oxygenated blood, an arterial catheter configured for insertion into an artery that supplies oxygenated blood, a monitoring device configured to monitor cardiac function of the patient, and a pumping device in electrical communication with the monitoring device, the pumping device being configured to siphon blood from the artery via the arterial catheter and continuously pump it into the vein via the venous catheter, the pumping device further being configured to inflate the venous catheter balloon in accordance with cardiac function information provided by the monitoring device such that the balloon is inflated in a counter-cardiac manner only during diastole to thereby urge oxygenated blood through the vein during diastole.

In one embodiment, a method for performing counter-cardiac retrograde perfusion comprises monitoring patient cardiac function and urging oxygenated blood through a patient vein in a counter-cardiac cycle.

### BRIEF DESCRIPTION OF THE DRAWINGS

The disclosed systems and methods can be better understood with reference to the following drawings. The components in the drawings are not necessarily to scale.

FIG. 1 is a schematic view of an embodiment of a system for performing counter-cardiac retrograde perfusion.

FIG. 2 is a block diagram of an embodiment of a pumping device shown in FIG. 1.

FIG. 3 is a flow diagram that illustrates an embodiment of a method for performing counter-cardiac retrograde perfusion.

FIG. 4A is an example electrocardiogram of a patient.

FIG. 4B is a plot of an example arterial blood pressure as a function of time for the patient of FIG. 4A.

FIG. 4C is a plot of an example blood pumping sequence used during counter-cardiac retrograde perfusion for the patient of FIGS. 4A and 4B.

FIG. 5 is a flow diagram that illustrates an embodiment of a method for performing counter-cardiac retrograde cerebral perfusion.

FIG. 6A is a first embodiment of a balloon catheter that can be used in the method of FIG. 5.

FIG. 6B illustrates use of the balloon catheter of FIG. 6A during counter-cardiac retrograde cerebral perfusion.

FIG. 7A is a second embodiment of a balloon catheter that can be used in the method of FIG. 5.

FIG. 7B illustrates use of the balloon catheter of FIG. 7A during retrograde cerebral perfusion.

FIGS. 8A and 8B are views of a balloon of a catheter during diastole and systole, respectively.

FIG. 9 is a flow diagram that illustrates an embodiment of a method for performing counter-cardiac retrograde renal perfusion.

FIG. 10A is an embodiment of a balloon catheter that can be used in the method of FIG. 9.

FIG. 10B illustrates use of the balloon catheter of FIG. 10A during counter-cardiac retrograde renal perfusion.

FIG. 11 is a flow diagram that illustrates an embodiment of an alternative method for performing counter-cardiac retrograde perfusion.

FIG. 12 illustrates two variants of a first embodiment of a balloon catheter that can be used in the method of FIG. 11.

FIG. 13A is a second embodiment of a balloon catheter that can be used in the method of FIG. 11.

FIGS. 13B and 13C illustrate use of the balloon catheter of FIG. 13A during diastole and systole, respectively, during counter-cardiac retrograde renal perfusion.

### DETAILED DESCRIPTION

As identified above, retrograde profusion is useful in preserving the health of organs such as the brain, but current techniques have attendant disadvantages. As described in the following, however, retrograde perfusion can be performed in a manner in which such disadvantages are avoided. In particular, retrograde perfusion can be performed in a pulsatile, counter-cardiac manner to avoid current perfusion disadvantages. In such counter-cardiac retrograde perfusion, oxygenated blood is provided to the venous system associated with a given organ in a pulsatile manner such that perfusion only or primarily occurs during diastole. When blood is supplied in this manner, overpressurization of the capillaries that link the arterial system and venous system of the organ is avoided. Moreover, drainage of blood from the organ is enabled, thereby avoiding organ edema.

Disclosed herein are embodiments of systems and methods that facilitate counter-cardiac retrograde perfusion. Although particular embodiments are disclosed, these embodiments are provided for purposes of example only to facilitate description of the disclosed systems and methods. Accordingly, other embodiments are possible.

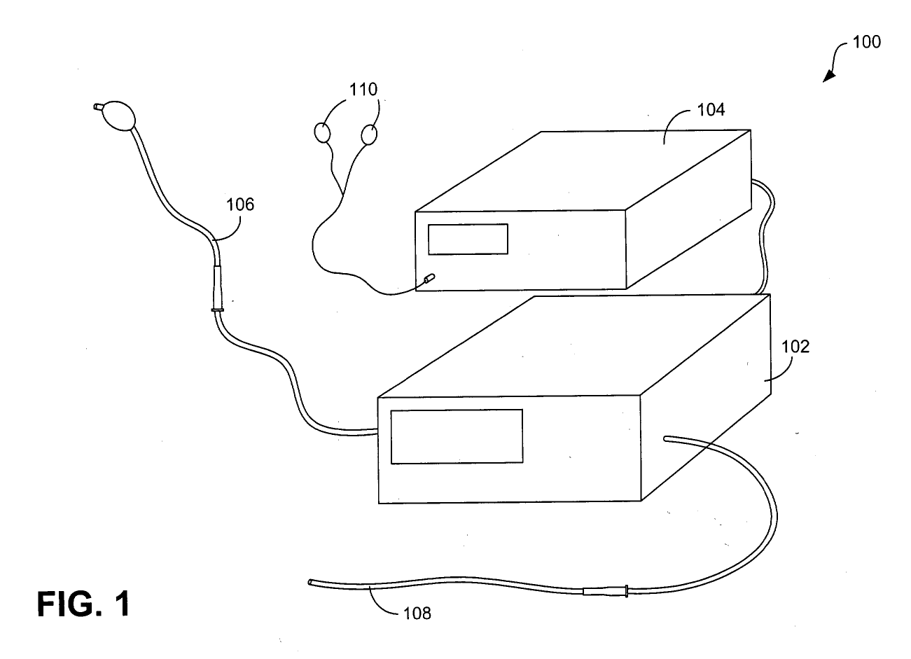

Referring now in more detail to the drawings, in which like numerals indicate corresponding parts throughout the several views, FIG. 1 illustrates an embodiment of a counter-cardiac retrograde perfusion system 100. As indicated in this figure, the system 100 generally comprises a pumping device 102, a patient monitoring device 104 a venous (delivery) catheter 106, and an arterial (supply) catheter 108. As is described in greater detail below, the pumping device 102 is configured to siphon oxygenated blood from the patient's arterial system and deliver the oxygenated blood to the venous system associated with an organ that is to be supplied with the blood.

The patient monitoring device 104 is in electrical communication with the pumping device and is configured to monitor cardiac function and, more particularly, the diastole/systole cycle. In one embodiment, the monitoring device 104 measures the electrical activity of the heart and, therefore, provides the functionality of a electrocardiograph machine. In such a case, the monitoring device 104 may use a patient interface comprising electrical sensors 110 that collect electrical data from the patient. In another embodiment, the monitoring device 104 alternatively, or in addition measures the blood pressure within the arterial system. In yet a further embodiment, the monitoring device 104 alternatively or in addition measures the flow of blood through the patient's arterial or venous system. In either of the latter two embodiments, the patient interface comprises a catheter or other lumen that is in fluidic communication with a patient artery or vein. In any case, however, the data collected by the monitoring device 104 is provided as an input to the pumping device 102 and, therefore, may be used to regulate operation of the pumping device.

As illustrated in the embodiment of FIG. 1, the venous catheter 106 is configured as a balloon catheter that includes a balloon that is inflated and deflated as desired to alternately obstruct and enable flow through the vein in which the catheter is used. In some embodiments, the venous catheter 106 comprises a self-inflating balloon catheter. In other embodiments, the venous catheter 106 comprises a manually-controlled balloon catheter in which inflation and deflation of one or more balloons are controlled by the pumping device 102. Specific example embodiments for the venous catheter 106 are described in the following. Typically, however, the venous catheter 106, as well as the arterial catheter 108, is constructed of medical grade polyvinyl chloride (PVC) plastic or other inert material.

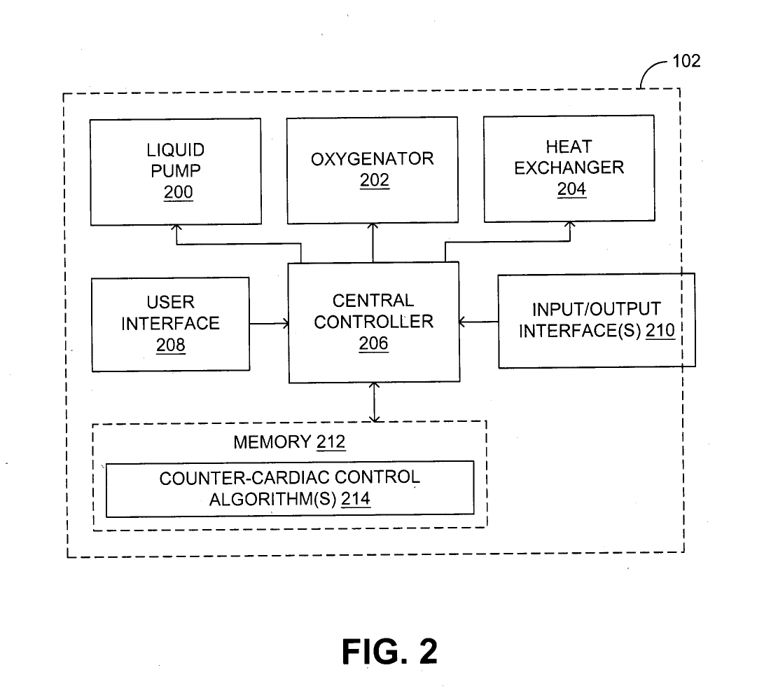

FIG. 2 illustrates an embodiment of the pumping device 102 shown in FIG. 1. As depicted in FIG. 2, the pumping device 102 can comprise a liquid pump 200 that is used to deliver oxygenated blood from a patient artery to a patient vein, an oxygenator 202 that is used to additionally oxygenate the blood obtained from the artery, a heat exchanger 204 that is used to heat or cool the blood obtained from the artery, and a central controller 206 that controls operation of the liquid pump, the oxygenator (when provided), and the heat exchanger (when provided).

The liquid pump 200 comprises any pump that is capable of drawing oxygenated blood from the patient artery and supplying it, in adequate volume and sufficient pressure, to the patient vein. By way of example, the liquid pump 200 comprises a peristalic (roller) pump or a centrifugal pump.

The oxygenator 202, when provided, adds oxygen to the blood and therefore enables the removal of carbon dioxide from the blood. By way of example, the oxygenator 202 comprises a bubble oxygenator that “bubbles” oxygen through the siphoned blood, or a membrane oxygenator that oxygenates the blood through contact with a system of membranes or fibers.

The heat exchanger 204, when provided, adds heat to or removes heat from the blood siphoned from the patient artery prior to its delivery to the patient vein. By way of example, the heat exchanger 204 comprises a plate or tube heat exchanger that creates a boundary between the siphoned blood and water that circulates through the heat exchanger.

The central controller 206 acts as the “brain” of the pumping device 102. The controller 206 may comprise a processing device such as a general-purpose processor, a microprocessor, one or more application-specific integrated circuits (ASICs), a plurality of suitably configured digital logic gates, and other well known electrical configurations comprised of discrete elements both individually and in various combinations to coordinate the overall operation of the pumping device 102.

In addition to the above-described components, the pumping device 102 can comprise a user interface 208 and one or more an input/output (I/O) interfaces 210, both of which may be used to control operation of the central controller 206. The user interface 208 comprises one or more buttons or keys of a device control panel (not shown) that a physician or technician may use to control operation of the pumping device 102. The I/O interface 210 receives inputs from the patient monitoring device 104 to control, inter alia, the manner in which oxygenated blood is pumped into the patient vein. In particular, received from the monitoring device 104 is cardiac function information that can be used to ensure delivery of oxygenated blood to a patient vein during diastole.

Operation of the central controller 206 is further affected by one or more counter-cardiac control algorithms 214 stored within device memory 212. By way of example, a counter-cardiac control algorithm 214 provides instructions to the central controller 206 as to when to actuate the liquid pump 200 in accordance with the monitored patient cardiac cycle. Although counter-cardiac operation of the central controller 206, and therefore the pumping device 102, is described as being performed in accordance with a counter-cardiac control algorithm (i.e., code) stored in memory, the same functionality can be obtained with appropriate hardware associated with or incorporated into the central controller.

Although not indicated in FIG. 2, the pumping device 102 typically further includes a blood reservoir that is used to store oxygenated blood siphoned from the patient artery to prevent unintended pumping of air or other gas into the patient vein a situations in which the supply of blood from the patient artery is interrupted. In addition, the pumping device 102 may comprise inlets that allow the addition of fluids or medications to the blood targeted for delivery to the organ in question.

Various code has been described herein. This code can be stored on any computer-readable medium for use by or in connection with any computer-related system or method. In the context of this document, a computer-readable medium is an electronic, magnetic, optical, or other physical device or means that contains or stores a computer program for use by or in connection with a computer-related system or method. These programs can be embodied in any computer-readable medium for use by or in connection with an instruction execution system, apparatus, or device, such as a computer-based system, processor-containing system, or other system that can fetch the instructions from the instruction execution system, apparatus, or device and execute the instructions.

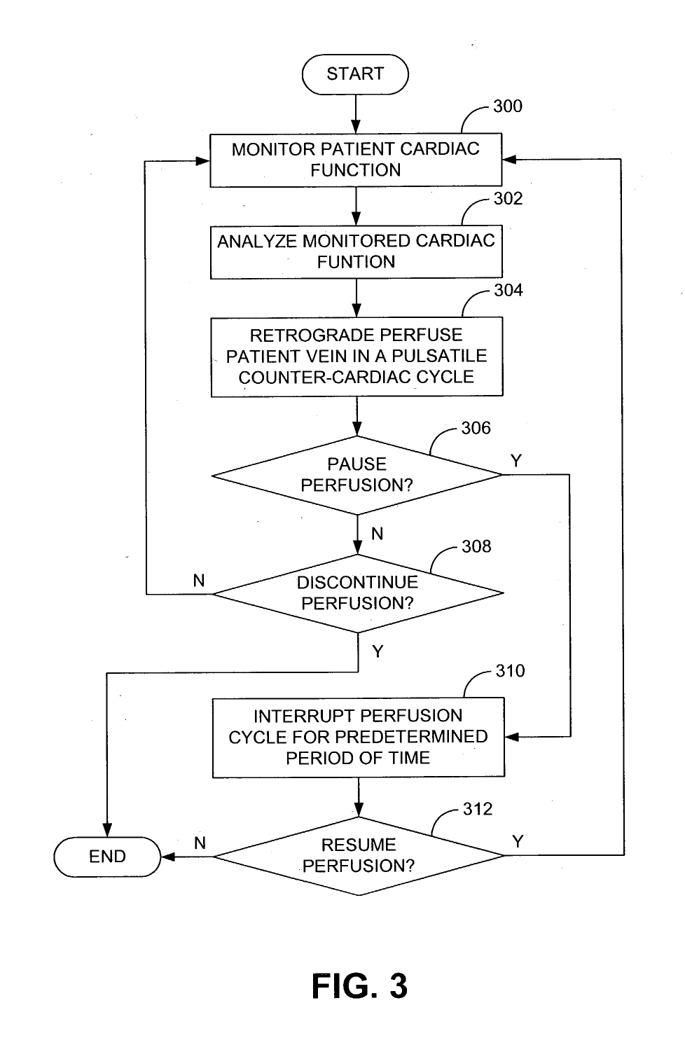

FIG. 3 is a flow diagram of an embodiment of a method for practicing counter-cardiac retrograde perfusion. In this and other flow diagrams of the present disclosure, process steps or blocks may represent modules, segments, or portions of code that include one or more executable instructions for implementing specific logical functions or steps in the process. In addition, although particular example process steps are described, alternative implementations are feasible. Moreover, steps may be executed out of order from that shown or discussed, including substantially concurrently or in reverse order, depending on the functionality involved.

Prior to initiation of the system, the venous catheter is inserted into the vein in which oxygenated blood is to be delivered, and the arterial catheter is inserted into the artery from which the oxygenated blood is to be collected. In each case, insertion is accomplished by a percutaneous stick using an appropriate rigid needle or trocar, or via cutdown or open incisions. A guide wire can be passed through the vein and/or artery under fluoroscopic monitoring to ensure that the wire is positioned correctly. Once the wire is guided through the vein and/or artery, the given catheter is passed over the guide wire so as to place the catheter in the correct position within the vein and/or artery. At this point, the guide wire is removed and the catheter may be secured in place by suturing the catheter to the patient's skin. Once both the venous catheter and the arterial catheter have been positioned in the manner described above, the pumping device is operated to siphon oxygenated blood from the artery into the blood reservoir.

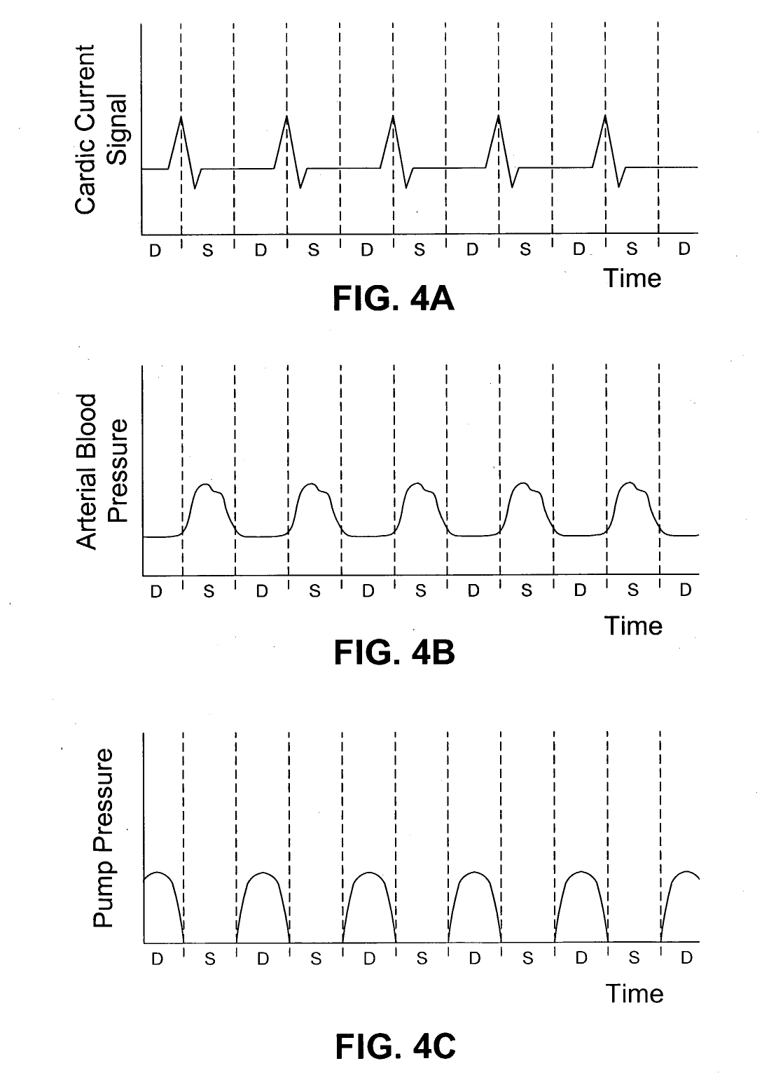

Prior or contemporaneous to the siphoning of oxygenated blood from the artery, the patient monitoring device monitors patient cardiac function, as indicated in block 300 of FIG. 3. As noted above, cardiac function monitoring can take several different forms. Two examples of such monitoring are illustrated in FIGS. 4A and 4B. With reference to FIG. 4A, the electrical signals that cause contraction of the heart are monitored to produce an electrocardiogram (EKG). From this electrocardiogram, the operation, i.e. pumping, of the heart can be identified. Therefore, the diastolic (D) and systolic (S) phases of the cardiac cycle can be determined. With reference to FIG. 4B, arterial blood pressure can also or alternatively be monitored to produce a plot of blood pressure as a function of time. This blood pressure can be measured from, for example, arteries located in the patient's wrist (e.g., radial artery) or groin (e.g., femoral artery). From this plot, the diastolic (D) and systolic (S) phases of the cardiac cycle can also be determined. Other forms of patient monitoring can be practiced to make this determination. For example, the arterial or venous blood flow as a function of time can be monitored.

Irrespective of the manner in which cardiac function is monitored, the monitored cardiac function is analyzed, as indicated in block 302 of FIG. 3. In particular, cardiac function is evaluated to determine the cyclical occurrence of diastole. Next, with reference to block 304, the oxygenated blood siphoned from the patient artery is retrograde perfused into the patient vein, via the venous catheter, in a pulsatile, counter-cardiac cycle. Specifically, blood is delivered in a reverse direction of the vein during diastole to prevent over-pressurization of patient capillaries intermediate the patient venous system and arterial system. In addition to this blood, appropriate pharmacologic agents, such as anticoagulation medications (e.g., heparin), may also be provided to the vein.

As is discussed in greater detail below, such counter-cardiac retrograde perfusion can be accomplished in various different ways. In a first technique, oxygenated blood is pumped into the vein only during diastole. Such a technique is illustrated in FIG. 4C, which shows the intermittent pulses of the pump occurring during diastole. In such a case, the balloon of the venous catheter is inflated prior to and during pumping of oxygenated blood (i.e., during diastole), and is deflated when prior to and during the period in which such pumping does not occur (i.e., during systole). In such a case, the balloon may comprise a self-inflating balloon that inflates when pressurized by the flow of oxygenated blood through the catheter.

In another technique, oxygenated blood is continuously pumped through the venous catheter and pulsatile, counter-cardiac flow within the vein is created by inflation and deflation of the catheter balloon, resulting in the same pressurization (and therefore flow) indicated in FIG. 4C. As is described below, inflation and deflation of the balloon can be effected by delivering to and drawing from the balloon a gas, such a helium.

Irrespective of the manner in which counter-cardiac retrograde perfusion is achieved, oxygenated blood is passed through the vein in a cyclical manner dependent upon heart operation. As illustrated in FIG. 4C, such delivery of blood can occur in a one-to-one ratio in which blood is forced through the vein during each consecutive diastole phase. Alternatively, however, blood can be delivered through the vein in another ratio. For example, blood can be forced through the vein during every other diastolic phase, during every third diastole phase, and so forth depending upon the volume of oxygenated blood to be provided to the organ and the net flow within the vein that is desired. In any case, blood is permitted to drain in the normal direction of flow within the vein during systolic phases of the cardiac cycle.

Perfusion in the manner described above may be paused periodically to enable greater drainage of blood from the organ being serviced, and therefore prevent edema. Therefore, counter-cardiac retrograde perfusion may be practiced for a predetermined period of time (e.g., 15 seconds) and then paused for another predetermined period of time (e.g., another 15 seconds) in an alternating fashion. Accordingly, with reference to decision block 306, it is determined whether perfusion is to be paused. If not, flow for the method continues to decision block 308 at which it is determined whether retrograde perfusion is to be discontinued all together. For example, if a procedure (e.g., surgical operation) for which retrograde perfusion was necessary has been completed, perfusion may be discontinued. If so, flow for perfusion session is terminated. If retrograde perfusion is to be continued, however, flow returns to block 300 described above.

With reference back to decision block 306, if retrograde perfusion is to be paused, flow continues to block 310 at which the perfusion cycle is interrupted for a predetermined period of time. After expiration of the predetermined period of time, flow for the method continues to decision block 312 at which it is determined whether retrograde perfusion is to be resumed. If not, flow for the perfusion session is terminated. If, on the other hand, retrograde perfusion is to be resumed, flow returns to block 300 described above.

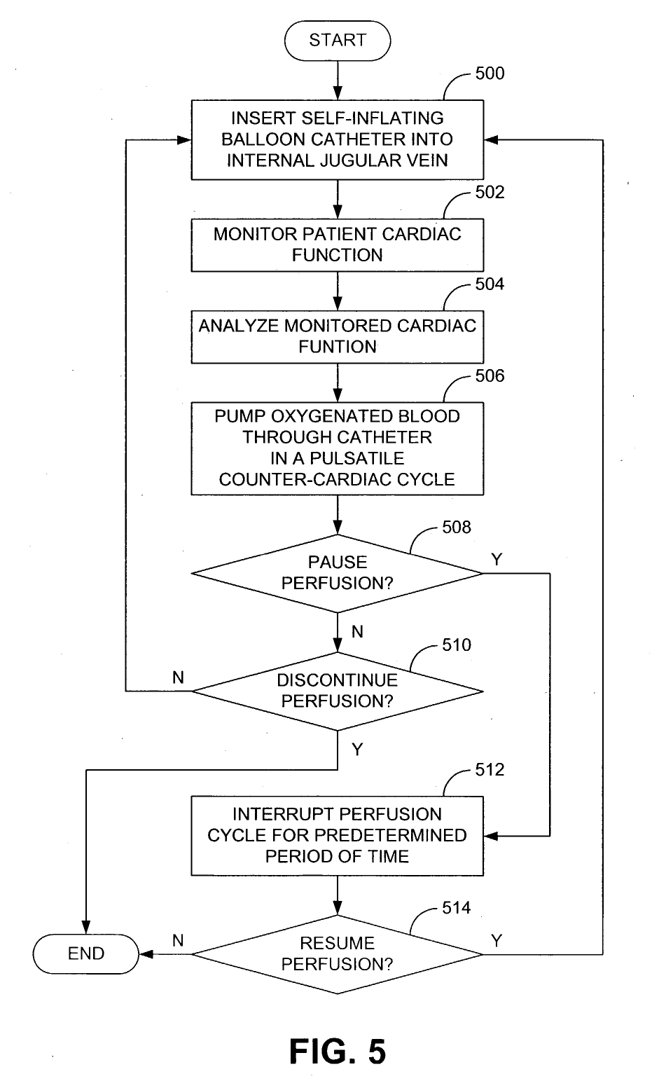

FIG. 5 is a flow diagram of another embodiment of a method for practicing counter-cardiac retrograde perfusion. More particularly, illustrated is an embodiment of a method for practicing counter-cardiac retrograde cerebral perfusion. In this case, a self-inflating balloon catheter is inserted into the internal jugular vein of the patient. The internal jugular vein used may depend upon the part of the brain that is being denied oxygen. For example, if a right-side carotid artery is blocked, the right internal jugular vein may be used to ensure that oxygenated blood reaches the right side of the brain.

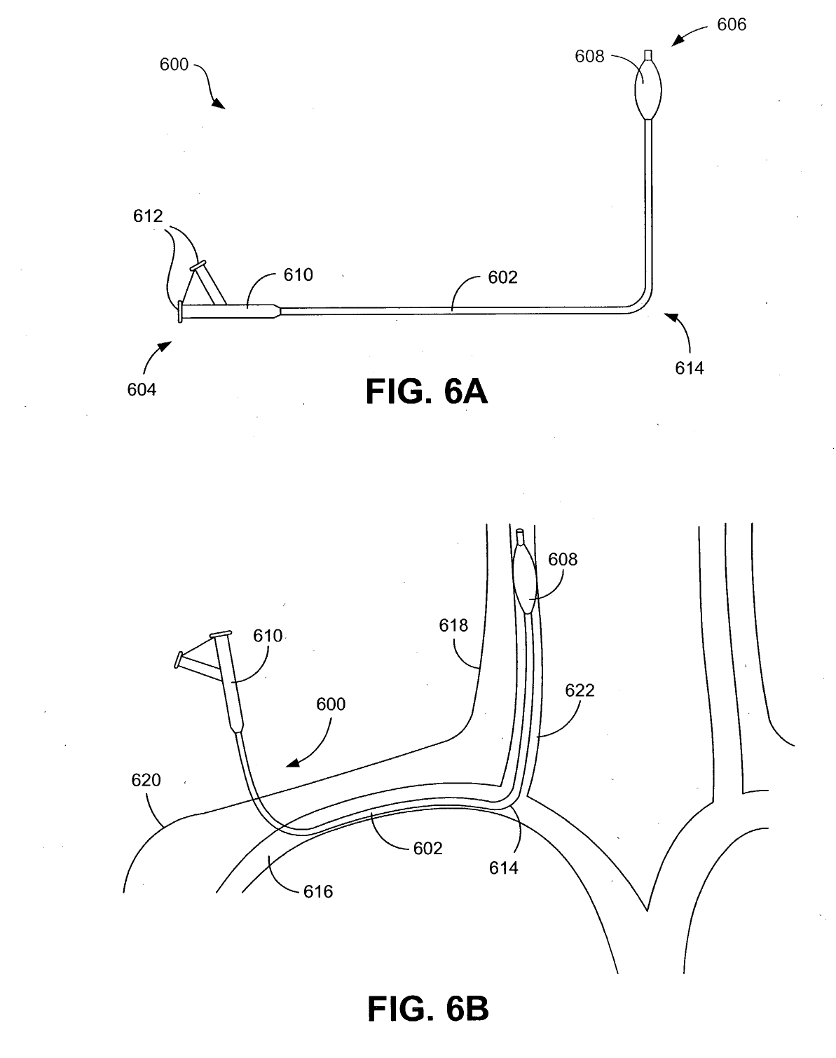

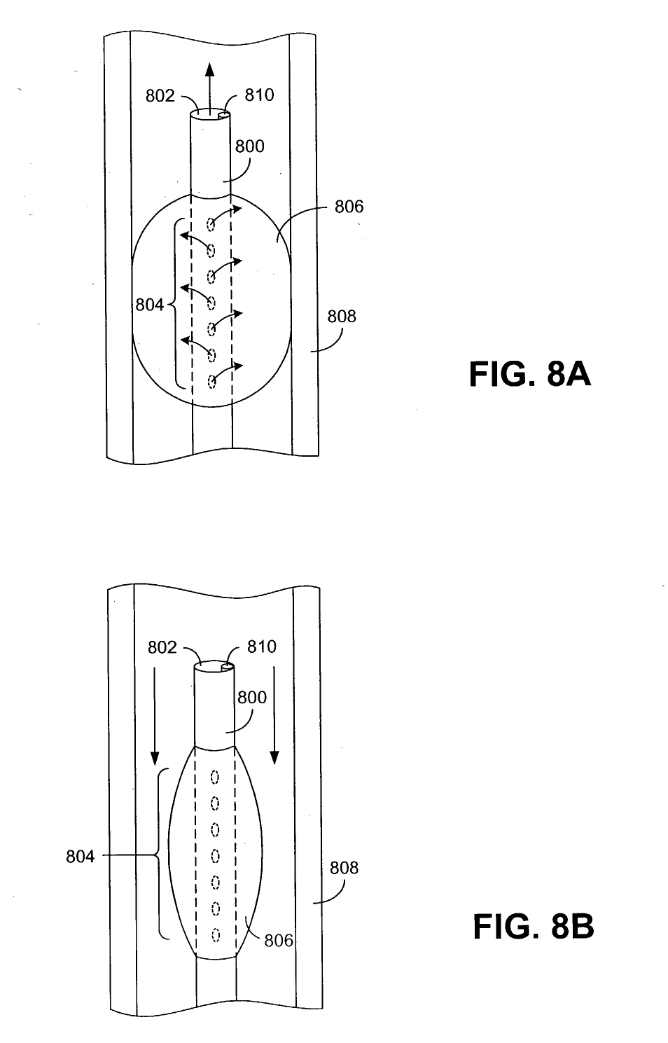

Insertion and fixation of the balloon catheter may be facilitated through use of a balloon catheter that is specifically configured for use in the internal jugular vein. FIG. 6A illustrates one such catheter. As indicated in this figure, the catheter 600 includes an elongated lumen 602 having a proximal end 604 and a distal end 606. Adjacent the distal end 606 of the lumen 602 is a pliable, self-inflating balloon 608. Apertures (not visible in FIG. 6A; see FIGS. 8A and 8B) formed through the walls of the lumen 602 within the portion of the lumen that is encapsulated by the balloon 608 are used to fill the balloon when blood is pumped through the lumen from its proximal end 604.

Positioned at the proximal end 604 of the lumen 602 is a coupler 610 that is used to connect the catheter 600 to the pumping device so that oxygenated blood from the pumping device can be passed through the catheter. As indicated in FIG. 6A, the coupler 610 includes at least one port 612 through which fluid may pass. One port 612 is placed in fluidic communication with the interior of the lumen 602 such that fluid (e.g., blood) passing through that port flows through the lumen. Another port 612, when provided, is in fluidic communication with an internal lumen (not visible in FIG. 6A; see FIGS. 8A and 8B) that may be used to measure pressure at the distal end of the catheter 602 during perfusion.

As is further indicated in FIG. 6A, the catheter 600 includes an angulation 614, such as a sharp bend or a kink, that facilitates insertion and fixation of the catheter. In the example of FIG. 6A, this angulation 614 is positioned at a central portion of the lumen 602 and comprises an approximately ninety degree bend that emulates the path traversed along the subclavian vein into the internal jugular vein. FIG. 6B illustrates such traversal. As indicated in this figure, the catheter 600 is inserted into the subclavian vein 616 through an incision made near between the patient's neck 618 and shoulder 620. Once passed into the subclavian vein 616, the catheter 600 is guided into the internal jugular vein 622. As shown in FIG. 6B, the subclavian vein 616 and the internal jugular vein 622 are at an approximately ninety degree angle with respect to each other. Therefore, the angulation of the catheter 600 aids in maneuvering the catheter from the subclavian vein 616 into the internal jugular vein 622 in that the catheter has a natural bias in the direction of the internal jugular vein when correctly guided to that vein. In addition, once the catheter 600 has been guided into the correct position for performing counter-cardiac retrograde perfusion, the angulation 614 helps maintains this positioning in that the catheter's angulation reflects the relative orientations of the subclavian vein 616 and the internal jugular vein 622. Notably, although access to the internal jugular vein 622 is acquired in FIG. 6B via the subclavian vein 616, the internal jugular vein can, alternatively, be directly accessed.

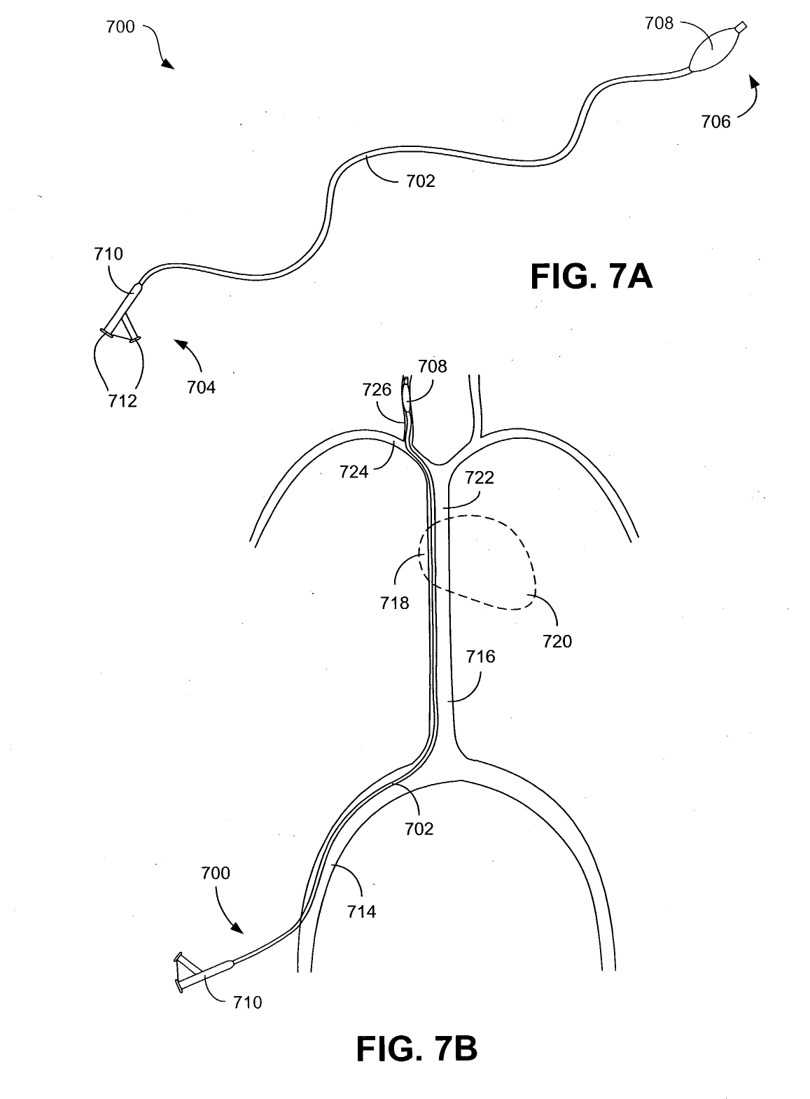

FIG. 7A illustrates another catheter 700 that may be used in counter-cardiac retrograde cerebral perfusion. As indicated in this figure, the catheter 700 also includes an elongated lumen 702 having a proximal end 704 and a distal end 706. In the embodiment of FIG. 7A, the catheter 700 is designed for insertion into the internal jugular vein via a femoral vein. Therefore, the lumen 702 is substantially longer than that of the catheter 600 shown in FIG. 6A.

Adjacent the distal end 706 of the lumen 702 is a pliable self-inflating balloon 708. The balloon 708 is configured such that it may be automatically expanded by the flow of blood through apertures (not visible in FIG. 7A; see FIGS. 8A and 8B) formed through the walls of the lumen 702 within the portion of the lumen that is encapsulated by the balloon 708.

Positioned at the proximal end 704 of the lumen 702 is a coupler 710 that is used to connect the catheter 700 to the pumping device. This coupler 710 also includes at least one port 712 through which fluid may pass. One port 712 is placed in fluidic communication with the interior of the lumen 702. Optionally, another port 712 is placed in fluidic communication with an internal lumen (not visible in FIG. 7A; see FIGS. 8A and 8B) used to measure pressure at the distal end of the catheter 702.

FIG. 7B illustrates use of the catheter 700. As illustrated in this figure, the catheter 700 is inserted into a femoral vein 714, up through the inferior vena cava 716, through the right atrium 718 of the heart 720, through the superior vena cava 722, through the subclavian vein 724, and finally into the internal jugular vein 726.

With reference to block 502 of FIG. 5, the patient monitoring device monitors patient cardiac function. As described above, cardiac function monitoring can comprise monitoring of one or more of electrical activity of the heart, arterial blood pressure, and arterial or venous blood flow. The monitored cardiac function is analyzed, as indicated in block 504 to determine the cyclical occurrence of diastole. Once this analysis is performed, oxygenated blood siphoned from the patient artery is pumped through the self-inflating balloon catheter in a pulsatile, counter-cardiac cycle, as indicated in block 506. In particular, blood is pumped in a reverse direction of the internal jugular vein during diastole to supply oxygenated blood to the brain.

During pumping, the balloon of the balloon catheter is inflated with the oxygenated blood. This phenomenon is illustrated in FIG. 8A. As depicted in this figure, when blood is pumped through the catheter 800, blood flows out through a distal opening 802 of the catheter. In addition, some of the pumped blood passes through apertures 804 formed in the portion of the catheter lumen that is encapsulated by the balloon 806. Because of the pliability of the balloon 806, the balloon expands as blood flows through the apertures 804 which, ultimately causes the balloon to contact the inner walls of the vein 808 in which the catheter 800 is positioned. As a consequence of this contact, blood that normally flows through the vein (in the downward direction in FIG. 8A) is impeded, thereby facilitating retrograde flow of the oxygenated blood.

As is further illustrated in FIG. 8A, a pressure port 810 may be located at the distal opening 802 of the catheter 800. When so provided, the port 810 is in fluidic communication with an internal lumen that is used to measure pressure within the vein 808 at the distal end of the catheter.

With reference next to FIG. 8B, illustrated is deflation of the balloon 806 prior to and during systole. As shown in this figure, the balloon 806 deflates because, during systole, no blood is pumped through the catheter 800, and therefore through the apertures 804. Because of this deflation, blood may flow in the normal direction of the vein to drain blood away from the organ at issue (e.g., brain).

Irrespective of the manner in which counter-cardiac retrograde perfusion is achieved, oxygenated blood is delivered through the internal jugular vein in a cyclical manner dependent upon heart performance. Such perfusion may be performed for any given period of time. By way of example, this perfusion is performed until a surgical procedure to remove a blockage from a carotid artery has been performed or until risk factors associated with an impending stroke have subsided. As noted previously, perfusion may be paused periodically to enable greater drainage of blood from the organ (i.e., brain) being serviced. Accordingly, with reference to decision block 508 of FIG. 5, it is determined whether perfusion is to be paused. If not, flow for the method continues to decision block 510 at which it is determined whether retrograde perfusion is to be discontinued. If perfusion is to be discontinued, flow for the perfusion session is terminated. If, on the other hand, retrograde perfusion is to be continued, flow for the method returns to block 500 and continues in the manner described above.

With reference back to decision block 508, if retrograde perfusion is to be paused, flow continues to block 512 at which the perfusion cycle is interrupted for a predetermined period of time. After expiration of the predetermined period of time, flow for the method continues to decision block 514 at which it is determined whether retrograde perfusion is to be resumed. If not, flow for the perfusion session is terminated. Alternatively, if retrograde perfusion is to be resumed, flow returns to block 500 described above.

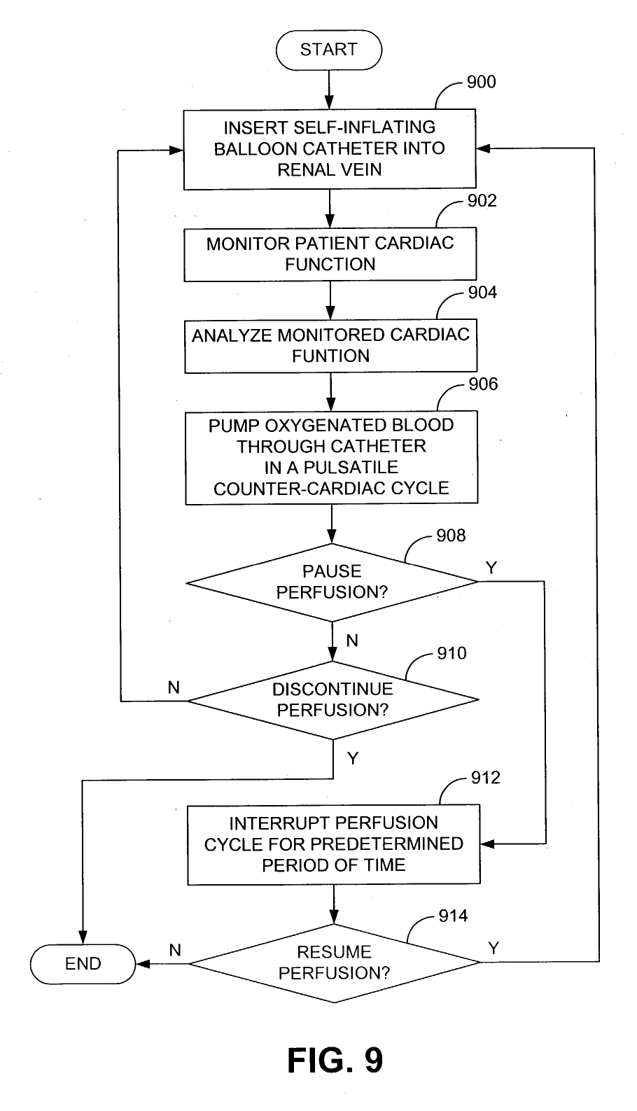

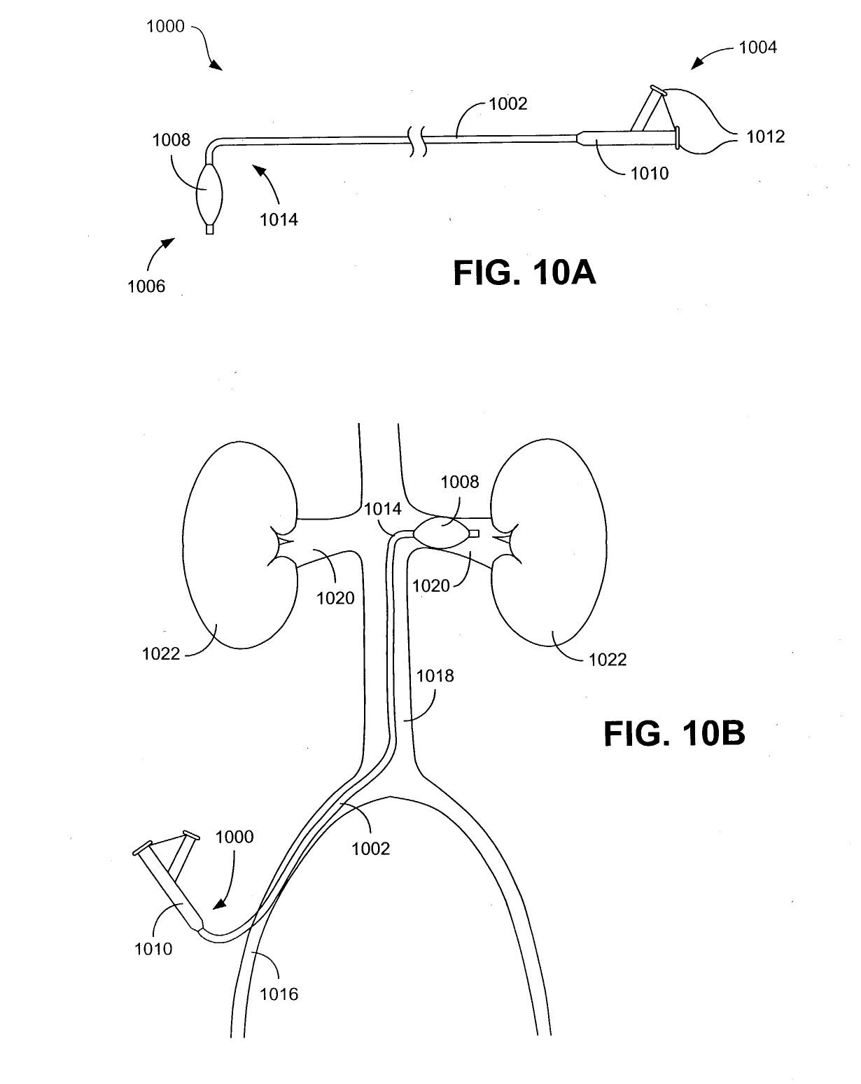

FIG. 9 is a flow diagram of yet another embodiment of a method for practicing counter-cardiac retrograde perfusion. More particularly, illustrated is an embodiment of a method for practicing counter-cardiac retrograde renal perfusion. In this case, a self-inflating balloon catheter is inserted into a renal vein of the patient. Beginning with block 900, a self-inflating balloon catheter is inserted into the renal vein. Insertion and fixation of the balloon catheter may be facilitated through use of a balloon catheter that is specifically configured for use in the renal vein. FIG. 10A illustrates one such catheter.

As indicated in FIG. 10, the catheter 1000 includes an elongated lumen 1002 having a proximal end 1004 and a distal end 1006. Adjacent the distal end 1006 of the lumen 1002 is a self-inflating balloon 1008 that, like other self-inflating balloons described above, is pliable so as to inflate when blood is pumped through the catheter 1000. Positioned at the proximal end 1004 of the lumen 1002 is a coupler 1010 that includes at least one port 1012. Again, one port 1012 is placed in fluidic communication with the interior of the lumen 1002 and another port, when provided, is in fluidic communication with an internal lumen (not visible in FIG. 10A) that is used to measure pressure at the distal end of 1006 the catheter 1002.

As is further indicated in FIG. 10A, the catheter 1000 includes an angulation 1014, such as a sharp bend or a kink, directly adjacent the balloon 1008 which facilitates insertion and fixation of the catheter. In the example of FIG. 10A, this angulation 1014 comprises an approximately ninety degree bend that emulates the path traversed along the inferior vena cava and into the renal vein. Positioning of the catheter 1000 within these veins is illustrated in FIG. 10B. As indicated in this figure, the catheter 1000 is inserted into the femoral vein 1016, along the inferior vena cava 1018, and into the renal vein 1020 associated with one of the patient's kidneys 1022. As shown in FIG. 10B, the inferior vena cava 1018 and the renal vein 1020 are at an approximately ninety degree angle with respect to each other. Therefore, the angulation 1014 of the catheter 1000 aids in maneuvering the catheter from the inferior vena cava 1018 into the renal vein 1020 and further aids in maintaining this placement.

With reference to block 902 of FIG. 9, the patient monitoring device monitors patient cardiac function. The monitored cardiac function is analyzed, as indicated in block 904 to determine the cyclical occurrence of diastole and, once this analysis is performed, oxygenated blood siphoned from the patient artery is pumped through the self-inflating balloon catheter in a pulsatile, counter-cardiac cycle, as indicated in block 906. In particular, blood is pumped in a reverse direction of the renal vein during diastole to supply oxygenated blood to the kidney.

During pumping, the balloon of the balloon catheter is inflated with the oxygenated blood. This phenomenon is illustrated in FIG. 10B. As depicted in this figure, when blood is pumped through the catheter 1000, blood flows through apertures (not shown) formed in the portion of the catheter lumen that is encapsulated by the balloon 1008. Because of the pliability of the balloon 1008, the balloon expands to make contact the inner walls of the renal vein 1008 in which the catheter 800 is positioned. As a consequence of this contact, blood that normally flows through the vein (to the left in FIG. 10B) is impeded, thereby facilitating retrograde flow of the oxygenated blood.

Through the performed counter-cardiac retrograde perfusion, oxygenated blood is delivered through the renal vein in a cyclical manner dependent upon heart performance. Such perfusion may be performed for any given period of time. By way of example, this perfusion is performed until a surgical procedure on the heart has been completed. After a given duration of perfusion, it is determined whether perfusion is to be paused, as indicated in decision block 908. If not, flow for the method continues to decision block 910 at which it is determined whether perfusion is to be discontinued. If perfusion is to be discontinued, flow for the perfusion session is terminated. If, on the other hand, retrograde perfusion is to be continued, flow for the method returns to block 900 and continues in the manner described above.

With reference back to decision block 908, if retrograde perfusion is to be paused, flow continues to block 912 at which the perfusion cycle is interrupted for a predetermined period of time. After expiration of the predetermined period of time, flow for the method continues to decision block 914 at which it is determined whether retrograde perfusion is to be resumed. If not, flow for the perfusion session is terminated. Alternatively, if retrograde perfusion is to be resumed, flow returns to block 900.

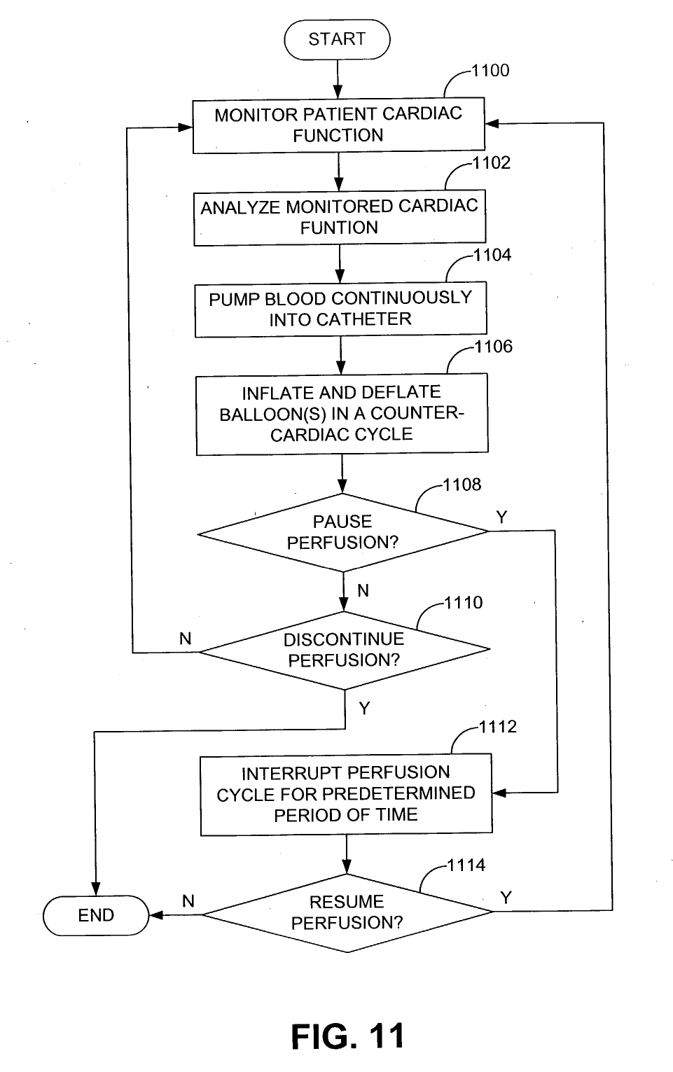

FIG. 11 is a flow diagram of a further embodiment of a method for practicing counter-cardiac retrograde perfusion. In this embodiment, oxygenated blood is continuously pumped through a balloon catheter, but pulsatilepulsatile, counter-cardiac flow is created by inflation and deflation of one or more catheter balloons. Beginning with block 1100 of FIG. 11, patient cardiac function is monitored and the monitored cardiac function is analyzed, as indicated in block 1102. At this time, blood is continuously pumped at a constant or fluctuating rate into the venous catheter, as indicated in block 1104. Simultaneous to this pumping or thereafter, one or more balloons of the venous catheter is/are inflated and deflated in a cyclical manner dependent upon heart performance, as indicated in block 1106. Specifically, the balloon(s) is/are inflated just before and during diastole and deflated just before and during systole. Inflation causes the blood provided by the catheter to pass in a retrograde direction along the vein and, therefore, supply the organ that the vein serves with oxygenated blood.

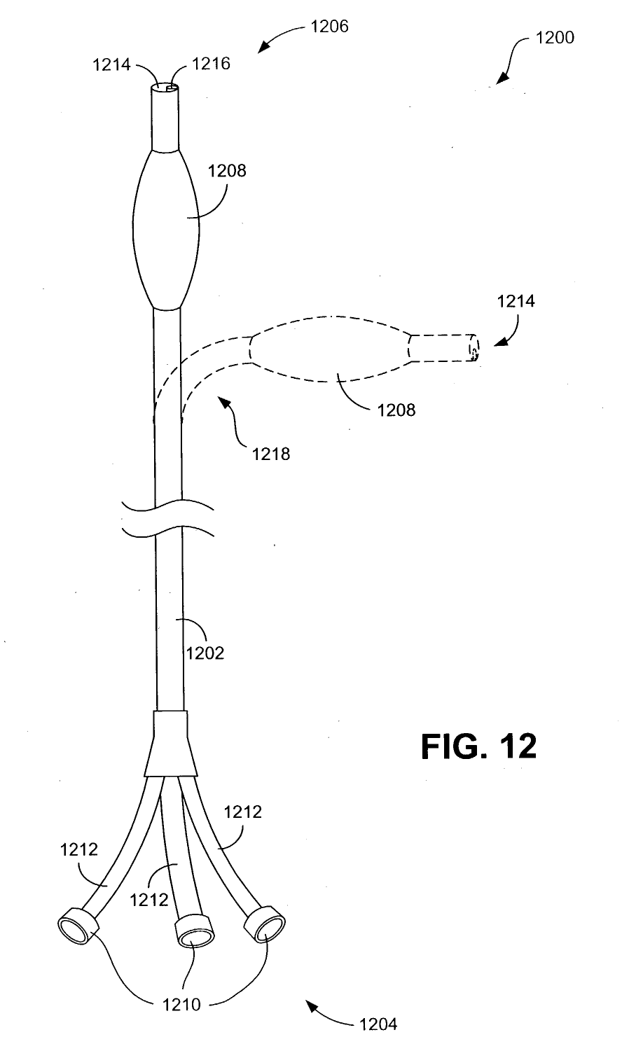

In that the catheter balloon(s) are not inflated automatically during pumping of blood, the catheter is configured to enable manually-controlled balloon inflation and deflation. FIG. 12 illustrates two variants of an embodiment of a manually-controlled balloon catheter 1200. As indicated in FIG. 12, the catheter 1200 includes an elongated outer lumen 1202 having a proximal end 1204 and a distal end 1206. Adjacent the distal end 1206 of the lumen 1202 is a manually-controlled balloon 1208. At the proximal end 1204 of the catheter 1200 is a coupling that comprises several ports 1210 and their associated lumens 1212 that, for example, pass through the outer lumen 1202. One of the lumens 1212 is in fluidic communication with the interior of the outer lumen 1202 such that blood pumped into the lumen 1212 flows through the outer lumen and out through an opening 1214 at the distal end 1206 of the catheter 1200.

Another of the lumens 1212 extends along the length of the catheter 1200 so as to have a port 1216 positioned at the distal end 1206 of the catheter, which can be used to measure pressure within the patient vein during perfusion. The other lumen 1212 extends to the balloon 1208 and is in fluidic communication with an interior space of the balloon. With this arrangement, that lumen 1212 can be used to pump gas (e.g., helium) into and out of the balloon 1208 to control its inflation and deflation.

As is depicted in dashed lines in FIG. 12, one variation the catheter 1200 further includes an angulation 1218 that facilitates insertion and fixation of the catheter relative to the patient venous system. This angulation 1218 can comprise a sharp (e.g., substantially ninety degree) bend or kink that, for instance, is positioned directly adjacent the balloon 1208 (FIG. 12) or at another position along the length of the outer lumen 1202 (not shown).

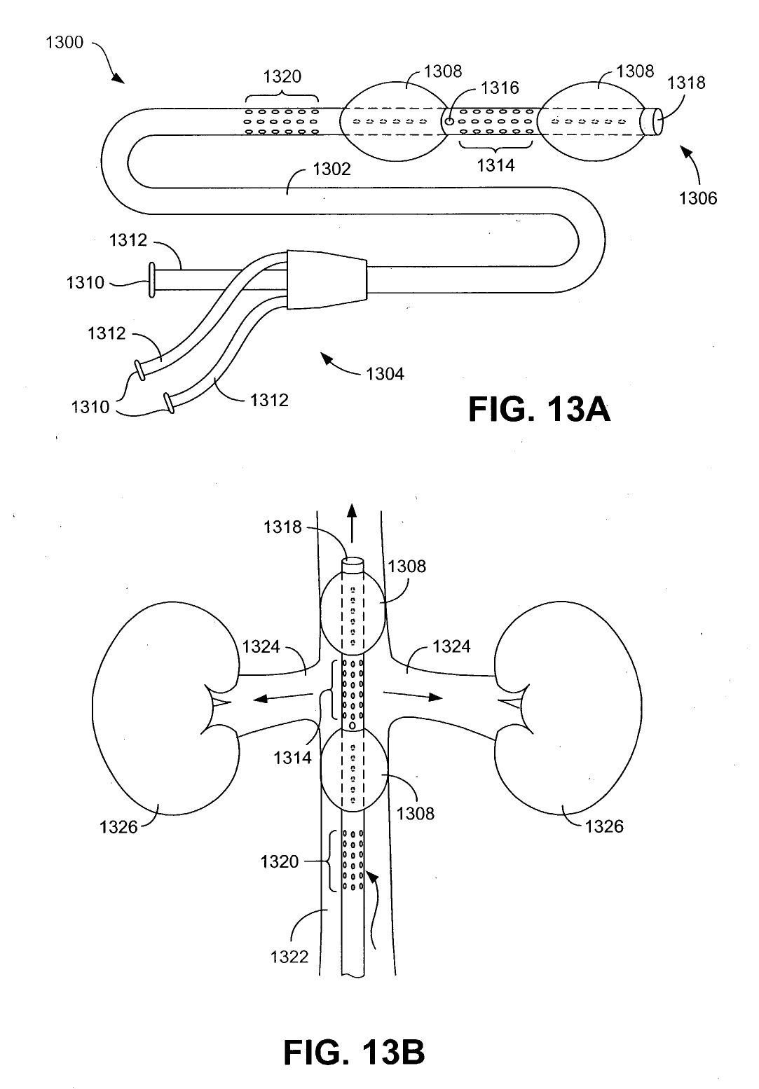

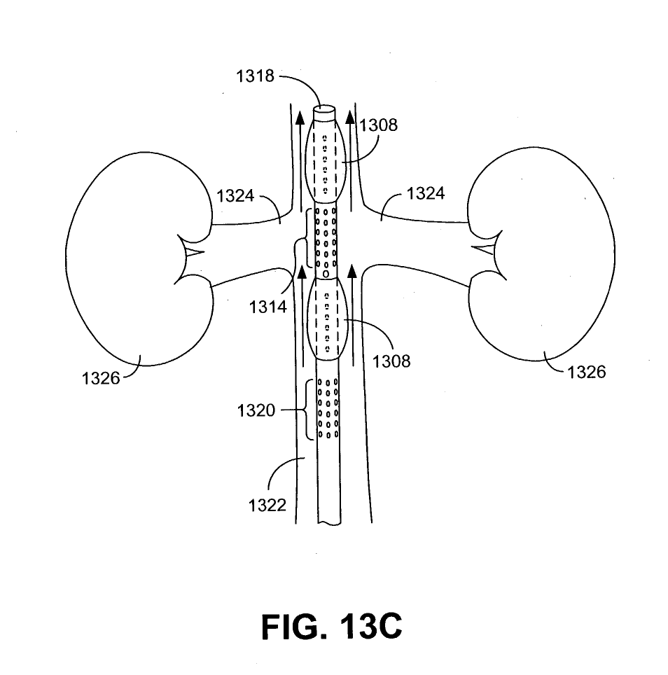

FIG. 13A illustrates a further embodiment of a manually-controlled balloon catheter 1300. This catheter 1300 is particularly suited for counter-cardiac retrograde renal perfusion. As indicated in FIG. 13, the catheter 1300 includes an elongated outer lumen 1302 having a proximal end 1304 and a distal end 1306. Generally adjacent the distal end 1306 of the lumen 1302 are two manually-controlled balloons 1308. At the proximal end 1304 of the catheter 1300 is a coupling that comprises several ports 1310 and their associated lumens 1312. These lumens 1312 include a primary lumen that is in fluidic communication with a group of apertures 1314 located along the outer lumen between the balloons 1308. Therefore, if blood is pumped into the primary lumen 1312, it flows through the catheter 1300 and out through the apertures 1314 to supply oxygenated blood to the patient in the vicinity of the renal veins.

Another of the lumens 1312 extends through the outer lumen 1302 to a port 1316 positioned between the two balloons 1308. This port 1316 is used to measure pressure in the vicinity of the renal veins during perfusion. The other lumen 1312 extends to both of the balloons 1308 and is in fluidic communication with an interior spaces of the balloons such that gas (e.g., helium) can be pumped into and out of the balloons to control their simultaneous inflation and deflation.

Also illustrated in FIG. 13A is an opening 1318 at the distal end 1306 of the catheter 1300. This opening 1318 is in fluidic communication with a further set of apertures 1320 located between the more proximal balloon 1308 and the proximal end 1304 of the catheter 1300. With this arrangement, blood that enters the apertures 1320 during diastole or systole can, in effect, flow through the catheter 1300, therefore the inferior vena cava, despite blockage of the inferior vena cava caused by inflation of the balloons 1308.

FIGS. 13B and 13C illustrate use of the catheter 1300 during diastole and systole, respectively. With reference first to FIG. 13B, oxygenated blood is pumped through the catheter 1300 and flows out the apertures 1314 located between the balloons 1308. Prior to and during diastole, the balloons 1308 are inflated so that normal blood flow through the inferior vena cava 1322 does not reach the renal veins 1324 and the kidneys 1326. Instead, this blood flows through the apertures 1320, through the catheter 1300, and out through the opening 1318 at the distal end 1306 of the catheter 1300. In this configuration, the renal veins 1324, and therefore the kidneys, are isolated from the venous system without interrupting normal blood flow through the inferior vena cava 1322. In addition, both kidneys 1326 can be retrograde perfused simultaneously with a single catheter instead of two.

Next, with reference to FIG. 13C, the balloons 1308 are deflated just prior to and during systole so as to permit a greater amount of blood to flow in the normal direction through the inferior vena cava 1322, as well as the renal veins 1324. During this time, the flow of oxygenated blood through the catheter 1300 can continue at the same rate at which it flowed during diastole. In alternative embodiments, however, this flow can be turned off or reduced in sequence with the deflation of the balloons 1308.

As in previously-described embodiments, perfusion may be paused periodically to enable greater drainage of blood from the organ being serviced, and therefore prevent edema. Therefore, with reference to decision block 1108, it is determined whether perfusion is to be paused. If not, flow for the method continues to decision block 1110 at which it is determined whether retrograde perfusion is to be discontinued. For example, if a surgical procedure for which retrograde perfusion was necessary has been completed, perfusion may be discontinued. If so, flow for perfusion session is terminated. If retrograde perfusion is to be continued, however, flow continues back to block 1100 described above.

With reference back to decision block 1108, if retrograde perfusion is to be paused, flow continues to block 1112 at which the perfusion cycle is interrupted for a predetermined period of time. After expiration of the predetermined period of time, flow for the method continues to decision block 1114 at which it is determined whether retrograde perfusion is to be resumed. If not, flow for the perfusion session is terminated. If, on the other hand, retrograde perfusion is to be resumed, flow returns to block 1100 described above.

While particular embodiments have been disclosed in detail in the foregoing description and drawings, it will be understood by those skilled in the art that variations and modifications thereof can be made without departing from the scope of the inventions set forth in the following claims. For example, in any of the described systems and methods, pharmacologic agents may be administered during perfusion by adding such agents to the oxygenated blood before it is pumped into the patient's vein.

1. A method for performing retrograde perfusion, comprising:
monitoring patient cardiac function; and
urging oxygenated blood through a patient vein in a counter-cardiac cycle.

2. The method of claim 1, wherein monitoring patient cardiac function comprises monitoring electrical activity of the patient's heart.

3. The method of claim 1, wherein monitoring patient cardiac function comprises monitoring arterial blood pressure.

4. The method of claim 1, wherein monitoring patient cardiac function comprises monitoring blood flow.

5. The method of claim 1, wherein urging oxygenated blood through a patient vein comprises urging oxygenated blood through the vein in a pulsatile, counter-cardiac retrograde manner such that the blood is urged through the vein in a reverse direction only during diastole.

6. The method of claim 5, wherein urging oxygenated blood through the vein in a pulsatile, counter-cardiac retrograde manner comprises pumping oxygenated blood into the vein in a pulsatile, counter-cardiac manner.

7. The method of claim 5, wherein urging oxygenated blood through the vein in a pulsatile, counter-cardiac retrograde manner comprises continuously pumping oxygenated blood into the vein and inflating a balloon of a catheter inserted into the vein in a pulsatile, counter-cardiac manner.

8. The method of claim 5, wherein urging oxygenated blood through the vein in a pulsatile, counter-cardiac retrograde manner comprises urging blood through the vein in a one-to-one ratio in which blood is urged through the vein during each consecutive diastole phase.

9. The method of claim 5, wherein urging oxygenated blood through the vein in a pulsatile, counter-cardiac retrograde manner comprises urging blood through the vein in less than a one-to-one ratio such that blood is not urged through the vein during each consecutive diastole phase.

10. The method of claim 1, further comprising pausing perfusion for a predetermined period of time to allow blood to drain from an organ served by the vein.

11. The method of claim 10, further comprising resuming perfusion after passage of the predetermined period of time.

12. The method of claim 11, further comprising alternately pausing and resuming perfusion in a cyclical manner.

13. A system for performing retrograde perfusion on a patient, comprising:
a venous catheter configured for insertion into a vein that serves an organ to be supplied with oxygenated blood;
an arterial catheter configured for insertion into an artery that supplies oxygenated blood;
a monitoring device configured to monitor cardiac function of the patient; and
a pumping device in electrical communication with the monitoring device, the pumping device being configured to siphon blood from the artery via the arterial catheter and pump it into the vein via the venous catheter, wherein blood is pumped into the vein in accordance with cardiac function information provided by the monitoring device such that blood is pumped only during diastole.

14. The system of claim 13, wherein the venous catheter is a self-inflating balloon catheter.

15. The system of claim 13, wherein the cardiac function information provided by the monitoring device is information about electrical activity of the patient's heart.

16. The system of claim 13, wherein the cardiac function information provided by the monitoring device is information about arterial blood pressure.

17. The system of claim 13, wherein the cardiac function information provided by the monitoring device is information about blood flow.

18. The system of claim 13, wherein the pumping device comprises a blood pump and a central controller that controls actuation of the pump.

19. The system of claim 18, wherein the central controller executes instructions stored in pumping device memory.

20. The system of claim 19, wherein the pumping device memory comprises at least one counter-cardiac control algorithm.

21. A system for performing retrograde perfusion on a patient, comprising:
a venous catheter configured for insertion into a vein that serves an organ to be supplied with oxygenated blood;
an arterial catheter configured for insertion into an artery that supplies oxygenated blood;
a monitoring device configured to monitor cardiac function of the patient; and
a pumping device in electrical communication with the monitoring device, the pumping device being configured to siphon blood from the artery via the arterial catheter and continuously pump it into the vein via the venous catheter, the pumping device further being configured to inflate the venous catheter balloon in accordance with cardiac function information provided by the monitoring device such that the balloon is inflated in a counter-cardiac manner only during diastole to thereby urge oxygenated blood through the vein during diastole.

22. The system of claim 21, wherein the venous catheter is a self-inflating balloon catheter.

23. The system of claim 21, wherein the cardiac function information provided by the monitoring device is information about electrical activity of the patient's heart.

24. The system of claim 21, wherein the cardiac function information provided by the monitoring device is information about arterial blood pressure.

25. The system of claim 21, wherein the cardiac function information provided by the monitoring device is information about blood flow.

26. The system of claim 21, wherein the pumping device comprises a blood pump and a central controller that controls actuation of the pump.

27. The system of claim 26, wherein the central controller executes instructions stored in pumping device memory.

28. The system of claim 27, wherein the pumping device memory comprises at least one counter-cardiac control algorithm.

29. A catheter, comprising:
a lumen having a proximal end and a distal end;
a self-inflating balloon adjacent the distal end of the lumen;
a coupler positioned at the proximal end of the lumen, the coupler including at least one port through which fluid may pass; and
a sharp bend formed in the lumen.

30. The catheter of claim 29, wherein the sharp bend is positioned at a central portion of the lumen.

31. The catheter of claim 29, wherein the sharp bend is formed directly adjacent the self-inflating balloon.

32. The catheter of claim 29, wherein the sharp bend forms an approximately ninety degree angle in the lumen.

33. A catheter, comprising:
a lumen having a proximal end and a distal end, the lumen having a length that enables it to extend from an adult femoral vein, through the vena cava, a subclavian vein, and into an internal jugular vein;
a self-inflating balloon adjacent the distal end of the lumen; and
a coupler positioned at the proximal end of the lumen, the coupler including at least one port through which fluid may pass.

34. A catheter, comprising:
a lumen having a proximal end and a distal end, the distal end of the lumen including an opening that opens to an interior of the lumen;
an inflatable balloon adjacent the distal end of the lumen;
a coupler positioned at the proximal end of the lumen, the coupler including two ports, a first port that is in fluidic communication with the lumen interior and the opening such that blood can be pumped through the first port, along the lumen, and out through the opening; and
an auxiliary lumen in fluidic communication with the second port and the inflatable balloon such that gas can be pumped through the second port, along the auxiliary lumen, and into the balloon to inflate it.

35. The catheter of claim 34, further comprising a sharp bend formed in the lumen.

36. The catheter of claim 35, wherein the sharp bend is positioned at a central portion of the lumen.

37. The catheter of claim 35, wherein the sharp bend is formed directly adjacent the balloon.

38. The catheter of claim 34, wherein the sharp bend forms an approximately ninety degree angle in the lumen.

39. A catheter, comprising:
a lumen having a proximal end and a distal end;
two inflatable balloons positioned along the lumen;
a set apertures formed through the lumen between the inflatable balloons;
an opening provided at the distal end of the lumen; and
a coupler positioned at the proximal end of the lumen, the coupler including a first port that is in fluidic communication with the apertures such that blood can be pumped through the first port and out through the apertures, and a second port in fluidic communication with the balloons such that gas can be pumped through the second port and into the balloons to inflate them.

40. The catheter of claim 39, further comprising a second set of apertures formed through the lumen between a proximal balloon and the proximal end of the lumen, the second set of apertures being in fluidic communication with the opening provided at the distal end of the lumen such that blood flowing through a vein in which the catheter is used may pass through the opening, along the catheter, and out through the second set of apertures of bypass the balloons.
